(19) United States
(12) Patent Application Publication
(10) Pub. No.: US 2024/0280475 A1
(43) Pub. Date: Aug. 22, 2024

(54) COLOR CHANGE SENSOR USING HOLLOW SILICA AND ION-PAIRING DYE CAPABLE OF DETECTING TRACE AMOUNTS OF ACETONE GAS

(71) Applicant: INDUSTRY-ACADEMIC COOPERATION FOUNDATION, YONSEI UNIVERSITY, Seoul (KR)

(72) Inventors: Wooyoung LEE, Seoul (KR); Jung Hyun KIM, Seoul (KR); Kyusik SHIN, Seoul (KR); Jae Jung PARK, Goyang-si (KR); Youngno KIM, Anyang-si (KR); Gaeun KIM, Seoul (KR); Jihyun LEE, Seoul (KR)

(21) Appl. No.: 18/396,665

(22) Filed: Dec. 26, 2023

(30) Foreign Application Priority Data
Feb. 16, 2023 (KR) 10-2023-0020919

(51) Int. Cl. G01N 21/29; G01N 33/00

(52) U.S. Cl. CPC G01N 21/29; G01N 33/0047

(57) ABSTRACT

The present disclosure relates to a color change dye prepared by impregnating hollow silica with a dye having a cationic dye and an anionic dye prepared using ion pairing, a sensor for detecting gas containing the above dye and a method for preparing the same, and more specifically to, a sensor that increases sensitivity and stability by increasing the area where the dye touches the gas and protecting the dye itself, since cationic dyes and anionic dyes are ion-paired to increase humidity resistance and react only with a trace amount of acetone. and the ion-paired dyes are then impregnated into hollow silica, and a method for preparing the same.

### CROSS-REFERENCE TO RELATED APPLICATION(S)

This application claims priority to Korean Patent Application No. 10-2023-0020919 filed in the Korean Intellectual Property Office on Feb. 16, 2023, the disclosure of which is incorporated by reference herein in its entirety.

### TECHNICAL FIELD

The present disclosure relates to a sensor for detecting trace amounts of acetone gas in exhaled gas with the naked eye without the aid of analysis equipment and a method for preparing the same, and more specifically, to a sensor that increases sensitivity and stability by increasing the area where the dye touches the gas and protecting the dye itself, since cationic dyes and anionic dyes are ion-paired to increase humidity resistance and react only with a trace amount of acetone. and the ion-paired dyes are then impregnated into hollow silica, and a method for preparing the same.

### DISCUSSION OF RELATED ART

Color change sensors, generally used in gas sensors, detect gas by changing color due to ring opening and closing reaction, ligand exchange, phases transition, and functional group change. These methods have the advantage that the reaction is possible at room temperature and the manufacturing cost of the sensor is low, but they generally have low sensitivity to trace low concentrations and require expensive instrument detection equipment. Further, it has the disadvantage of requiring a long reaction time and having weak resistance to humidity and temperature due to the use of pH dye.

Therefore, in order to improve sensitivity and resistance to humidity and temperature, research is being conducted to improve sensitivity by maximizing the surface area with polymer nano particles, silica nano particles, etc. and to increase resistance to humidity and temperature by impregnating these nanoparticles with pH dyes or modifying their surfaces.

However, these methods had difficulty in obtaining the desired level of sensitivity for detection substances in exhaled gas and had the disadvantage of lower sensitivity and stability compared to sensors using metal oxides or other methods. In addition, research is being conducted in various ways on sensors that detect hydrogen sulfide, carbon dioxide, nitrogen dioxide, etc. in exhaled gas. However, there is no sensor technology that can detect low concentration acetone gas by color change using pH dye and distinguish it with the naked eye without instrument detection.

Therefore, in order to detect trace amounts of acetone gas with the naked eye without the aid of analysis equipment, the present inventors used a dye that reacts with acetone vapor regardless of pH and two types of cationic and anionic dyes that change significantly with the naked eye due to the production of acid and used ion-paring technology to increase humidity resistance and manufacture dyes that react only with trace amounts of acetone, impregnates hollow silica with the dyes to increase the area in which the dyes touch the gas and protect the dyes themself, thereby confirming the increase of sensitivity and stability. Later, it was designed to enable connection with analysis equipment.

### SUMMARY

The present disclosure is to provide a sensor for detecting trace amounts of acetone gas in exhaled gas with the naked eye without the aid of analysis equipment and a method for preparing the same.

The present disclosure provides a color change dye for detecting acetone gas, in which the color change dye is prepared by mixing a cationic dye and an anionic dye to perform ion-paired dye and impregnating hollow silica with the ion-paired dye, and one of the cationic dye and the anionic dye is a dye that reacts to acetone vapor, and the other is a dye that reacts to acid production.

Further, the present disclosure provides a method of preparing a color change dye for detecting acetone gas, the method comprising: a first step of mixing and stirring a cationic dye and an anionic dye to prepare an ion-paired dye, wherein one of the cationic dye and the anionic dye is a dye that reacts to acetone vapor, and the other is a dye that reacts to acid production;

a second step of adding an amine group to the ion-paired dye; and a third step of impregnating hollow silica with the amine group-added ion-paired dye to prepare a color change dye.

Further, the present disclosure provides a sensor for detecting acetone gas, the sensor comprising the color change dye for detecting acetone gas according to the present disclosure.

Further, the present disclosure provides a method of detecting exposure to acetone gas by visually observing the color change of the dye using the sensor for detecting acetone gas according to the present disclosure.

The present inventors uses a dye that reacts with acetone vapor regardless of pH and two types of cationic and anionic dyes that change significantly with the naked eye due to the production of acid and uses ion-paring technology to increase humidity resistance and manufacture dyes that react only with trace amounts of acetone, impregnates hollow silica with the dyes to increase the area in which the dyes touch the gas and protect the dyes themself, thereby enhancing sensitivity and stability.

The dye manufactured according to the present disclosure can detect trace amounts of acetone gas with the naked eye without the help of analysis equipment, and the sensitivity can be further increased when the auxiliary role of the device or analysis equipment is added.

### BRIEF DESCRIPTION OF THE DRAWINGS

FIG. 1 is a view showing the results of an experiment to analyze the selectivity of a dye prepared according to an embodiment of the present disclosure to acetone gas, and specifically, FIG. 1 shows the color change when exposed to each gas of (a) air reference gas, (b) acetone 10 ppm, (c) ammonia 10,000 ppm, (d) H₂S 10 ppm, (e) H₂ 500 ppm, (f) isoprene 50 ppm, and (g) CO2 200 ppm for 10 minutes;

FIG. 2 is a view showing a microscopic image of hollow silica particles prepared according to an embodiment of the present disclosure, and specifically, FIG. 2 shows (a) single silica particle, (b) hollow silica particle, 50 nm (c) hollow silica particle, 70 nm, and (d) hollow silica particle, 100 nm;

FIG. 3 is a view showing images of hollow silica particles prepared according to an embodiment of the present disclosure and an acetone sensor prepared using the same, and specifically, FIG. 3 is a view showing (a) hollow silica particles and (b) a sensor exposed to acetone gas after being impregnated with an ion-paired dye;

FIG. 4 is a graph showing the color change rate results of dyes prepared according to an embodiment of the present disclosure according to changes in humidity, temperature, and time, and specifically, FIG. 4 is a graph showing the color change rate of the acetone sensor when (a) the humidity is 0 to 80%, (b) the temperature is 25 to 70° C., and (c) the time is 0 to 3 minutes;

FIG. 5 is a graph of the visual color change rate analysis of the sensor prepared according to an embodiment of the present disclosure over time (1 to 10 min) at 10 ppm of acetone and 10% humidity;

FIG. 6 is a graph of the visual color change rate analysis of the sensor prepared according to an embodiment of the present disclosure over time (1 to 10 min) at 5 ppm of acetone and 10% humidity; and

FIG. 7 is a graph of the visual color change rate analysis of the sensor prepared according to an embodiment of the present disclosure over time (1 to 10 min) at 1 ppm of acetone and 10% humidity.

### DETAILED DESCRIPTION

The terms used in this specification will be briefly described, and the present disclosure is described in detail.

Hereinafter, preferred embodiments of the present disclosure are described in detail. Terms or words used in this specification and patent claims should not be construed as limited to their usual or dictionary meanings but should be construed with meanings and concepts consistent with the technical details of the present disclosure.

The embodiments described in this specification are preferred embodiments of the present disclosure, and do not represent the entire technical idea of the present disclosure, so there may be various equivalents and modifications that can replace them at the time of filing the present application.

The present disclosure provides a color change dye for detecting acetone gas, in which the color change dye is prepared by mixing a cationic dye and an anionic dye to perform ion-paired dye and impregnating hollow silica with the ion-paired dye, and one of the cationic dye and the anionic dye is a dye that reacts to acetone vapor, and the other is a dye that reacts to acid production.

The dye that reacts to acetone vapor preferably includes anyone selected from the group consisting of methyl red, thymol blue, naphthol orange, and conge red, and more preferably includes methyl red.

The dye that reacts to acid production preferably includes anyone selected from the group consisting of meta cresol purple, brilliant green, thymol blue, cresol red, methyl orange, crystal violet. ethyl violet, and methyl violet, and more preferably includes one of meta cresol purple and brilliant green.

The hollow silica preferably has a particle size of 10 to 500 nm in diameter, and more preferably has a particle size of 50 to 100 nm.

Further, the present disclosure provides a method of preparing a color change dye for detecting acetone gas, the method comprising: a first step of mixing and stirring a cationic dye and an anionic dye to prepare an ion-paired dye, wherein one of the cationic dye and the anionic dye is a dye that reacts to acetone vapor, and the other is a dye that reacts to acid production;

a second step of adding an amine group to the ion-paired dye; and a third step of impregnating hollow silica with the amine group-added ion-paired dye to prepare a color change dye.

In the first step of the method, the dye that reacts to acetone vapor preferably includes anyone selected from the group consisting of methyl red, thymol blue, naphthol orange, and conge red, and more preferably includes methyl red.

In the first step of the method, the dye that reacts to acid production preferably includes anyone selected from the group consisting of meta cresol purple, brilliant green, thymol blue, cresol red, methyl orange, crystal violet. ethyl violet, and methyl violet, and more preferably includes one of meta cresol purple and brilliant green.

In the first step of the method, the stirring is preferably performed at 200 to 300 rpm for 10 to 15 hours and is more preferably performed at 240 to 260 rpm for 11 to 13 hours.

In the second step of the method, the amine group preferably includes one or more materials selected from the group consisting of polyethylene imine, hydroxylamine sulfate, and amino silane.

In the method, glycerol is preferably added.

The method may include:
1) adding a cationic dye and an anionic dye to a solvent, respectively, to prepare a cationic dye solution and an anionic dye solution;
2) mixing the cationic dye solution and the anionic dye solution, stirring them, and filtering the precipitate obtained from the stirring to obtain a solid dye; and
3) drying the solid dye, dissolving it in a solvent to prepare a solution, adding it to the solution in which a material containing an amine group is dissolved in the solvent, adding glycerol and stirring to prepare a color change dye.

In steps 1) to 3), the solvent may be a mixture of methanol and water in a ratio of 2:1 to 3:2, but is not limited thereto.

In step 2), the stirring is preferably performed at 200 to 300 rpm for 10 to 15 hours, and is more preferably performed at 240 to 260 rpm for 11 to 13 hours, but is not limited thereto.

In step of 3), the amine group preferably includes one or more materials selected from the group consisting of polyethylene imine, hydroxylamine sulfate, and amino silane, but is not limited thereto.

In step 3), the stirring is preferably performed at 200 to 300 rpm for 1 to 5 hours, and is more preferably performed at 240 to 260 rpm for 1 to 3 hours, but is not limited thereto, but is not limited thereto.

Further, the present disclosure provides a sensor for detecting acetone gas, the sensor comprising the color change dye for detecting acetone gas according to the present invention.

Further, the present disclosure provides a method of detecting exposure to acetone gas by visually observing the color change of the dye using the sensor for detecting acetone gas according to the present disclosure.

In addition to the color conversion dye according to the present disclosure, the sensor may further include any one or more of a support, an adhesive layer, a catalyst layer, or a protective layer.

The support may be a porous support and may be selected from the group consisting of graphene, carbon allotrope, ceramic oxide, and mixtures thereof.

The adhesive layer is used to adhere the color change dye to the support. Any material having adhesive ability may be used, and epoxy resin may be used, but is not limited thereto.

The catalyst layer can be made of any material that promotes the reaction of the color change dye, and a porous structure may be used, but is not limited thereto.

The protective layer can be made of any transparent or semi-transparent material to facilitate observation of the color change of the color conversion dye. polyethylene terephthalate (PET) or polyethylene (PE) may be used, but are not limited to thereto.

Hereinafter, preferred examples are presented to aid understanding of the present disclosure. the following examples are merely illustrative of the present disclosure, and it is clear to those skilled in the art that various changes and modifications are possible within the scope and spirit of the present disclosure. It is obvious that such variations and modifications fall within the scope of the attached patent claims.

### Example 1. Cationic Dye Selection

To identify dyes that react with acetone gas, a total of seven dyes (congo red, methyl red, naphthol orange, thymol blue, cresol red, brilliant green, and methyl green) were exposed to acetone gas.

As a result, as shown in Table 1 below, the three dyes cresol red, brilliant green, and methyl green did not react with acetone gas, but the four dyes methyl red (ΔE=38.3), thymol blue (ΔE=17.8), naphthol orange (ΔE=16.6), and conge red (ΔE=14.3) reacted with acetone gas. In particular, among the dyes that reacted to acetone gas, methyl red (ΔE=38.3) was selected as the dye with the largest ΔE of RGB change as a cationic dye.

### Example 2. Anionic Dye Selection

To select a dye that has the greatest color change when the pH is changed to acid due to sulfuric acid by utilizing the mechanism where acetone gas and primary amine group react to produce sulfuric acid, a total of 10 dyes (crystal violet, ethyl violet, methyl violet, cresol red, methyl orange, brome cresol green, meta cresol purple, brilliant green, alizarian red S, thymol blue) were used.

As a result, as shown in Table 2 below, brome cresol green did not react at all, and as a result of observing the color changes of the remaining 9 dyes, brilliant green (ΔE=52.9) and meta cresol purple (ΔE=42.1) were confirmed to be highly reactive and were selected as anionic dyes.

### Example 3. Preparation of Dye Using Ion Pairing Method

Ion pairing was performed using the cationic and anionic dyes selected in Examples 1 and 2.

Each of cationic dye and anionic dye was dissolved by adding 3% to a solvent mixed with methanol and water in a ratio of 3:2. Afterwards, each dye solution was stirred at 250 rpm using a mechanical stirrer for 12 hours, and after stirring, the precipitate was filtered through filter paper to obtain solid content.

After drying the obtained dye, 3% of the total volume of materials containing amine groups compared to the total volume (for example, polyethylene imine, hydroxylamine sulfate, amino silane) were dissolved in a solvent mixed with methanol and water in a ratio of 3:2. Afterwards, the solid content was dissolved in methanol at a rate of 0.15% compared to the total volume and added to the previous solution. 5% of glycerol was added compared to the total volume. Thus, Dye was prepared by stirring with a stirrer for about 2 hours.

### Example 4. Preparation of Hollow Silica

220 g of water was added to a 500 mL reactor. While stirring, the internal temperature was set to 60° C. Then, when the temperature was stabilized, 0.14 g of HNO₃ and 1.47 g of siliane (PTMS (Trimethoxyphenylsilane), N-2-(aminoethyl)-3-aminopropylmethyl dimethoxysilane, 3-aminopropyltriethoxysilane, 3-methacryloxypropyl triethoxysilane, 3-glycidoxypropyl trimethoxysilane) were added in that order.

After 60 seconds, 14.7 g of ammonia water and 14.7 g of methanol were sequentially added and stirred. After reacting at 270 rpm for 2 hours, 11.86 g of Na₂CO₃ aqueous solution as a coagulant was added and stirred again for 30 minutes, then taken out from the reactor and washed three times in a centrifuge at 1000 rpm for 10 minutes. At this time, the solvent was washed twice with methanol for the first time and with ethanol for the third time.

Then, sonication was performed at 60° C. for 60 minutes to obtain the final hollow silica after etching. At this time, in order to control the particle size according to the amount of silane added, 0.1%, 0.3%, 0.6%, and 0.8% of the total volume were added.

### Example 5. Preparation of Acetone Sensor

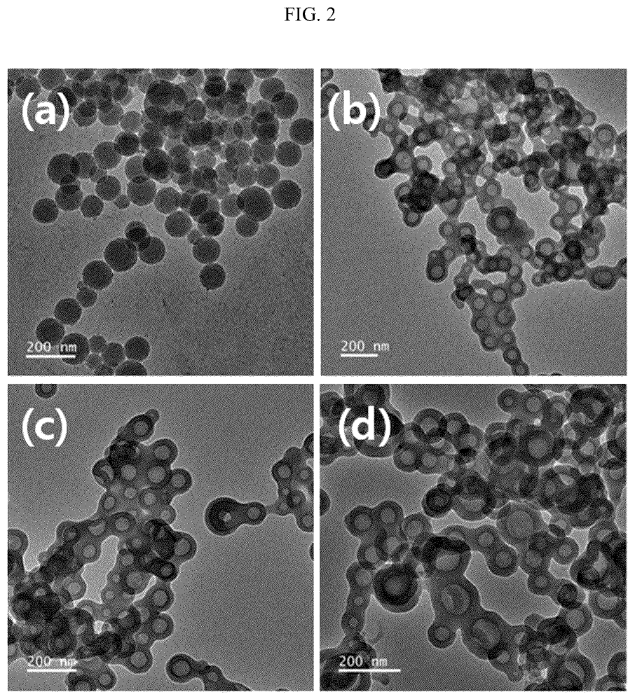

In order to increase the dye content and the exposed specific surface area of the gas, it was prepared from single silica particles to hollow silica particles as in Example 4, and it was confirmed that the size was also manufactured consistently (FIG. 2).

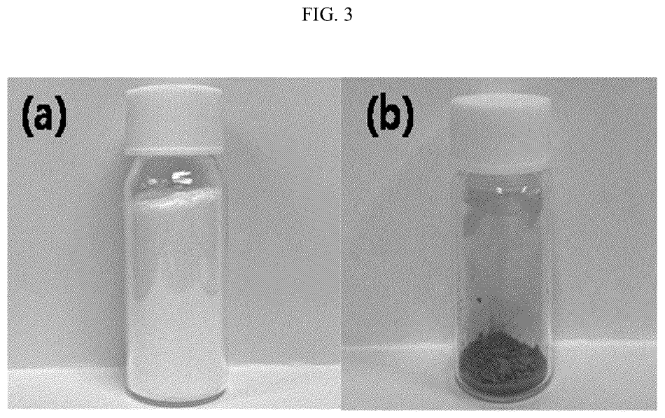

Among these, the smallest and most consistent 50 nm particles were selected, and an acetone sensor was manufactured through impregnation with ion paring dye (FIG. 3a).

It was confirmed that the color of the manufactured acetone sensor changed when exposed to acetone gas (FIG. 3b).

### Experimental Example 1. Analysis of Acetone Gas Selectivity of Ion-Paired Dye

To find out whether it is possible to selectively distinguish only acetone gas when preparing a color change sensor by dropping the ion-paired dye prepared in Example 3 on a silica sheet, color changes were analyzed through exposure to 5 different gases (ammonia, H₂S, H₂, isoprene, and CO₂).

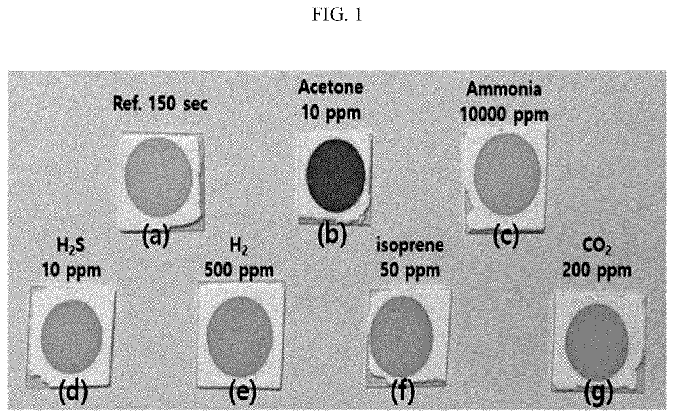

As a result, the gas that changes color for 150 seconds is 10 ppm of acetone. It was confirmed that there was no reaction at 10000 ppm of ammonia, 10 ppm of H₂S, 500 ppm of H₂, 50 ppm of isoprene, and 200 ppm of CO₂, and the color was the same as that of the reference (FIG. 1).

### Experimental Example 2. Analysis of Increase in Color Change and Increase in Humidity and Temperature Resistance Due to Increase in Surface Area of Hollow Silica

In general, when pH dyes are used, sensitivity decreases rapidly due to moisture, and sensitivity decreases rapidly even at humidity of less than 10%. To overcome this vulnerability to humidity, an acetone sensor was prepared as in Example 5.

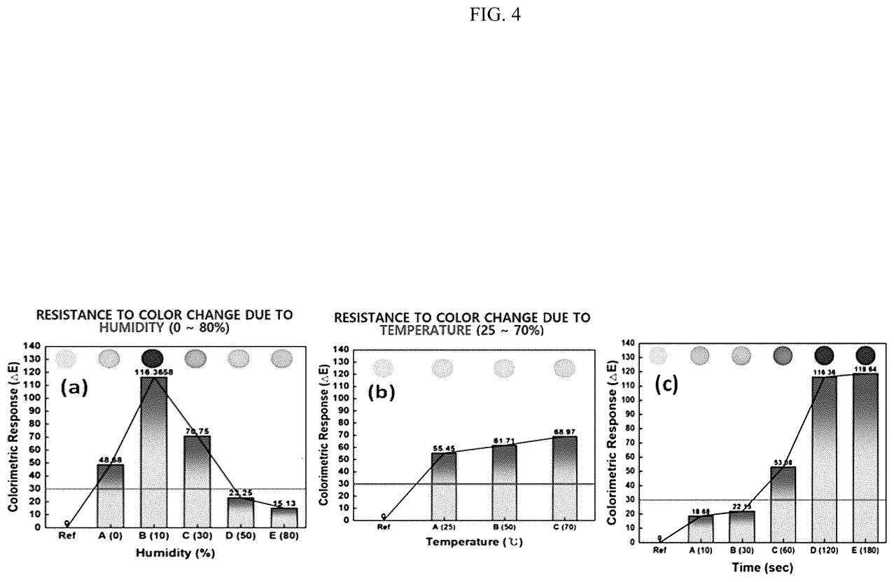

As a result of analysis using this acetone sensor, it was confirmed that color change was possible with the naked eye even in the presence of 10 ppm of acetone gas and 30% humidity, and it was found that the color change rate was maintained from 25 to 70° C. in temperature resistance (FIGS. 4a and 4b). At this time, it is determined that the reason for the slight increase in color change is because the activity of the gas increases. Further, it was confirmed that as time increases, the color change rate increases significantly, and the maximum color change occurred at 180 seconds of exposure (FIG. 4c).

### Experimental Example 3. Sensitivity Analysis of Color Change when Exposed to a Trace Amount of Acetone

The acetone sensor prepared in the same manner as in Example 5 was used to observe the color change by exposure to acetone gas depending on the concentration and time.

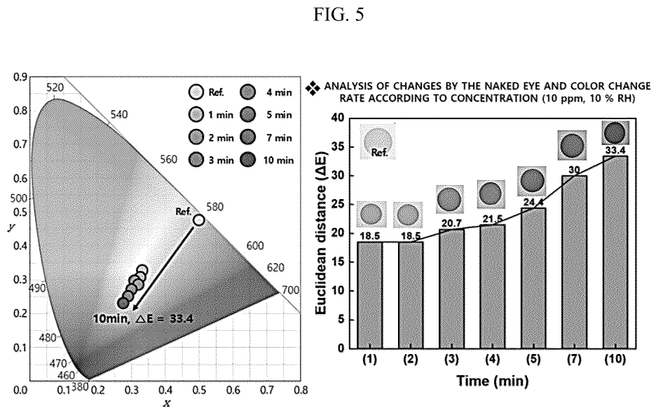

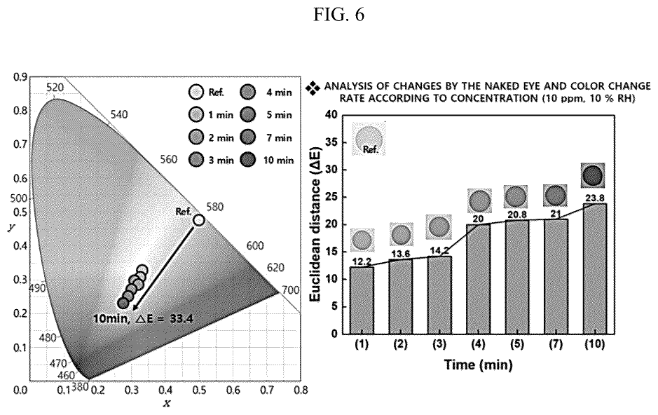

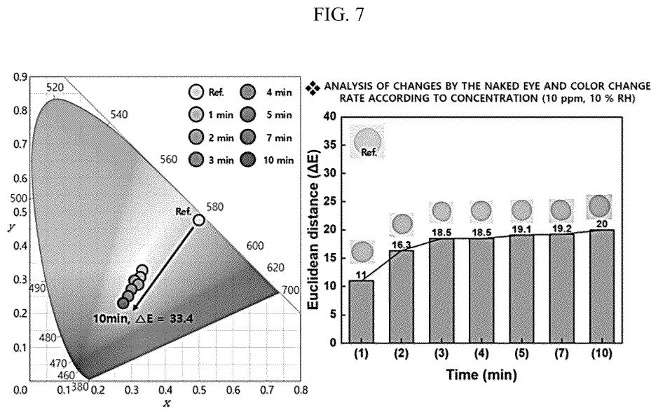

As a result of the analysis, it was expressed as ΔE by exposure to acetone concentrations of 1, 5, and 10 ppm for 10 minutes under 10% humidity conditions, and it was confirmed that color change occurred rapidly at high concentrations (FIGS. 5 to 7). However, it was confirmed that after exposure for 10 minutes, a clear color change occurred even at 1 ppm with the naked eye, confirming that acetone concentration up to 1 ppm could be detected at 10% humidity (FIG. 7).

The above description is only an example of the technical idea of the present embodiment, and various modifications and variations can be made to those skilled in the art without departing from the essential characteristics of the present embodiment. Therefore, the present disclosure is not intended to limit the technical idea of the present embodiment, but to explain, and the scope of the technical idea of the present disclosure is not limited by these embodiments. The scope of protection of the present disclosure should be interpreted according to the claims below, and all technical ideas within the scope equivalent thereto should be construed as being included in the scope of rights of the present disclosure.

What is claimed is:

1. A color change dye for detecting acetone gas, wherein the color change dye is prepared by mixing a cationic dye and an anionic dye to perform ion-paired dye and impregnating hollow silica with the ion-paired dye, and
wherein one of the cationic dye and the anionic dye is a dye that reacts to acetone vapor, and the other is a dye that reacts to acid production.

2. The color change dye of claim 1, wherein the dye that reacts to acetone vapor includes anyone selected from the group consisting of methyl red, thymol blue, naphthol orange, and conge red.

3. The color change dye of claim 1, wherein the dye that reacts to acid production includes anyone selected from the group consisting of meta cresol purple, brilliant green, thymol blue, cresol red, methyl orange, crystal violet. ethyl violet, and methyl violet.

4. The color change dye of claim 1, wherein the hollow silica has a particle size of 50 to 100 nm in diameter.

5. A method of preparing a color change dye for detecting acetone gas, the method comprising:
a first step of mixing and stirring a cationic dye and an anionic dye to prepare an ion-paired dye, wherein one of the cationic dye and the anionic dye is a dye that reacts to acetone vapor, and the other is a dye that reacts to acid production;
a second step of adding an amine group to the ion-paired dye; and
a third step of impregnating hollow silica with the amine group-added ion-paired dye to prepare a color change dye.

6. The method of claim 5, wherein in the first step, the dye that reacts to acetone vapor includes anyone selected from the group consisting of methyl red, thymol blue, naphthol orange, and conge red.

7. The method of claim 5, wherein in the first step, the dye that reacts to acid production includes anyone selected from the group consisting of meta cresol purple, brilliant green, thymol blue, cresol red, methyl orange, crystal violet. ethyl violet, and methyl violet.

8. The method of claim 5, wherein in the first step, the stirring is performed at 200 to 300 rpm for 10 to 15 hours.

9. The method of claim 5, wherein in the second step, the amine group includes one or more materials selected from the group consisting of polyethylene imine, hydroxylamine sulfate, and amino silane.

10. The method of claim 5, wherein in the second step, glycerol is further added.

11. A sensor for detecting acetone gas, the sensor comprising a color change dye for detecting acetone gas, wherein the color change dye is prepared by mixing a cationic dye and an anionic dye to perform ion-paired dye and impregnating hollow silica with the ion-paired dye, and
wherein one of the cationic dye and the anionic dye is a dye that reacts to acetone vapor, and the other is a dye that reacts to acid.

12. A method for detecting acetone gas, the method comprising:
exposing a sensor to an environment where the presence of acetone gas is suspected to observe the color change of the ion-paired dye is observed,
wherein the sensor is a sensor for detecting acetone gas, comprising a color change dye for detecting acetone gas, wherein the color change dye is prepared by mixing a cationic dye and an anionic dye to perform ion-paired dye and impregnating hollow silica with the ion-paired dye.
